(19) United States
(12) Patent Application Publication
(10) Pub. No.: US 2012/0058974 A1
(43) Pub. Date: Mar. 8, 2012

(54) Composition comprising pesticide and benzotriazole UV absorbers

(75) Inventors: Andrea Misske, Speyer (DE); Christian Bittner, Bensheim (DE); Michael Ishaque, Mannheim (DE); Markus Hoffmann, Barcelona (ES); Richard Riggs, Mannheim (DE); Ruediger Sens, Ludwigshafen (DE); Andre Kabat, Hassloch (DE); Sylke Haremza, Neckargemuend (DE); Douglas D. Anspaugh, Apex, NC (US); David M. Terry, Cary, NC (US)

(73) Assignee: BASF SE, Ludwigshsafen (DE)

(21) Appl. No.: 13/254,936

(22) PCT Filed: Mar. 10, 2010
(86) PCT No.: PCT/EP10/53003

§ 371 Date: Sep. 6, 2011

Related U.S. Application Data
(60) Provisional application No. 61/160,124, filed on Mar. 13, 2009.

(51) Int. Cl. A01N 37/34; A01N 65/12; A01P 3/00; A01P 7/04; A01P 13/00; A01N 25/22; C07D 249/20

(52) U.S. Cl. 514/66; 514/785; 514/788; 514/522; 548/261

(57) ABSTRACT

The present invention relates to an agrochemical composition comprising pesticide and UV absorber. The invention furthermore relates to UV-absorbers and to the use thereof in agrochemical compositions. It moreover relates to a method of controlling phytopathogenic fungi and/or undesirable plant growth and/or undesirable insect or mite infestation and/or of regulating plant growth.

The present invention relates to an agrochemical composition comprising pesticide and UV absorber. The invention furthermore relates to UV-absorbers and to the use thereof in agrochemical compositions. It moreover relates to a method of controlling phytopathogenic fungi and/or undesirable plant growth and/or undesirable insect or mite infestation and/or of regulating plant growth. Combinations of preferred features with other preferred features are included in the present invention.

Agrochemical compositions comprising pesticide and UV absorber are generally known:

WO 1992/03926 discloses insecticidal compositions comprising a pyrethroid, a UV absorption agent and an antioxidant.

EP 0 376 888 A1 discloses compositions for controlling harmful insects comprising a substance which modifies the behavior of the harmful insects and a pesticidally active compound, both of which are contained in a flowable matrix which protect the behavior-modifying substance from UV radiation. The compositions in general comprise from 51 to 98 wt. % of the matrix, which is conventionally a UV absorber which preferably has a viscosity of from 1,000 to 40,000 cp. A suitable UV absorber is, for example, a mixture of the following alkoxylated 2-(2-hydroxyphenyl)-benzotriazoles TinuA and TinuB

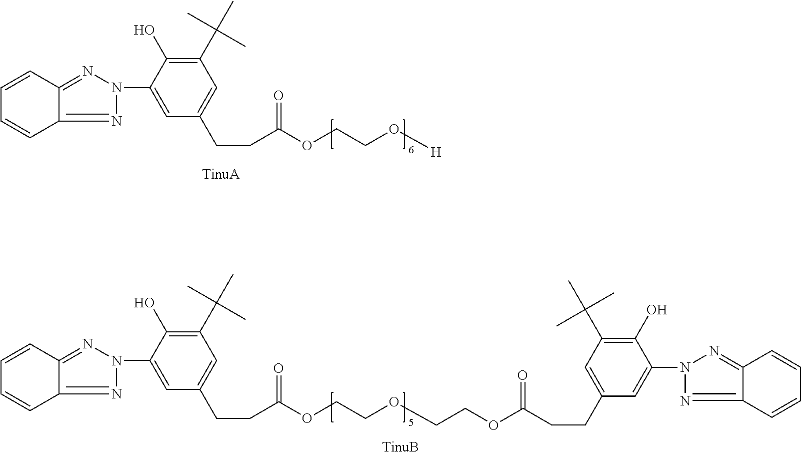

in a weight ratio of TinuA to TinuB of 50 to 38, which is obtainable under the trade name Tinuvin® 1130 from Ciba.

WO 2006/089747 discloses a method for protecting materials comprising the use of a composition comprising a capsule which contains a photolabile pesticide and a UV absorber such as alpha-[3-[3-(2H-benzotriazol-2-yl)-5-(1,1-dimethylethyl)-4-hydroxyphenyl]-1-oxopropyl]-omega-hydroxy-poly(oxy-1,2-ethanediyl).

WO 1997/42815 discloses a composition comprising 0.1 to 20% of pesticide, 0.01 to 30% of pheromone and 40 to 98% of UV absorber. Tinuvin 1130, for example, can be employed as the UV absorber.

WO 2008/085682 discloses a composition comprising a photolabile pesticide and a UV stabilizer, which can be, for example, a benzotriazole or a derivative thereof.

UV absorbers from the class of the benzotriazoles are known:

EP 0 280 650 discloses benzotriazoles of the following structure, where R can be for example —OCH₂CH₂OCH₂CH₂OC₂H₅ or —NHCH₂CH₂OC₂H₅.

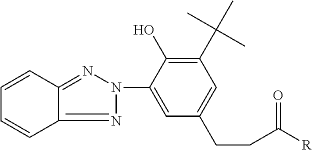

Benzotriazoles of the following structure (CAS No. 127519-17-9) are commercially available from Ciba under the trade names Tinuvin® 99 or Tinuvin® 384-2. In this structure, Alkyl stands for a mixture of branched and linear C7-9 alkyl groups.

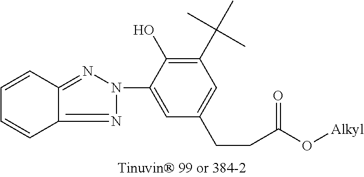

Benzotriazoles of the following structure (CAS No. 96478-09-0) are commercially available from Ciba under the trade name Tinuvin® R796.

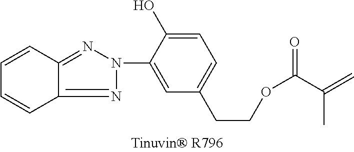

The UV absorbers, in particular from the class of benzotriazoles, from the prior art have various disadvantages: Further auxiliaries, such as antioxidants, must be added. The UV absorbers must be employed in a matrix. The UV absorbers have not been able to stabilize light-sensitive pesticides for a sufficiently long period of time.

The object of the present invention was to provide alternative UV absorbers from the class of benzotriazoles for use in agrochemical compositions. A further object was to discover benzotriazole UV absorbers which stabilize UV-sensitive pesticides. The stabilization should be better than in the prior art, in particular also at relatively low concentrations of benzotriazole UV absorber. The present invention also had for a subject to provide a benzotriazole UV absorber that has increased surface activity and is capable of reducing the surface tension of water to a greater extent. An object was furthermore to discover benzotriazole UV absorbers which allow simpler formulation of pesticides, for example in that fewer auxiliaries, such as surfactants, are necessary.

The object was achieved by an agrochemical composition comprising pesticide and UV absorber, wherein the UV absorber corresponds to the formula I

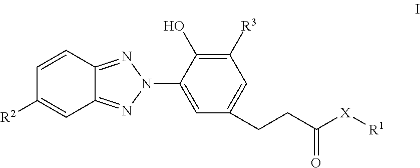

wherein
X: NH or O;
R¹: —[C₂-C₄ alkoxy]ₙ—(C₁-C₁₈ alkyl) or —[CH₂CH₂NH]ₙ—H;
R²: H or Cl;
R³: H or C₁-C₈ alkyl; and
n: 3 to 50.

Suitable UV absorbers are the structures of the formula I as defined above.

Suitable R¹ radicals are —[C₂-C₄ alkoxy]ₙ—(C₁-C₁₈ alkyl) or —[CH₂CH₂NH]ₙ—H, preferably —[C₂-C₄ alkoxy]ₙ—(C₁-C₁₈ alkyl) and more preferably —[CH₂CH₂O]ₙ—CHₛ.

The expression “C₂-C₄ alkoxy” preferably represents —CH₂CH₂O—, —CH(CH₃)CH₂O— or —CH(CH₂CH₃)CH₂O—, particularly —CH₂CH₂O—. Mixtures of —CH₂CH₂O—, —CH(CH₃)CH₂O— or —CH(CH₂CH₃)CH₂O— are likewise possible, in which case the alkoxy units can appear in random order or as blocks, preferably as blocks. Preference is given to mixtures of —CH₂CH₂O— and —CH(CH₃)CH₂O—, particularly mixtures of —CH₂CH₂O— and —CH(CH₃)CH₂O, which occur as blocks.

The expression “C₁-C₁₈ alkyl” typically represents a linear or branched, preferably linear, alkyl group having 1 to 18 carbon atoms.

In one preferred embodiment, the alkyl group is linear and comprises 1 to 8, more preferably 1 to 4 and particularly one carbon atom. In a further preferred embodiment, the alkyl group is branched and comprises 3 to 18, preferably 6 to 18, more preferably 9 to 15 and particularly 10 to 13 carbon atoms.

The expression “—CH₂CH₂NH—” is a general empirical formula and represents a monomer unit of the polyethyleneimine group [CH₂CH₂NH]ₙ. These polyethyleneimine groups can be linear or branched, and they are preferably branched. Branched polyethyleneimine groups conventionally contain primary, secondary and tertiary amino groups. The molar ratio of primary/secondary/tertiary amino groups can be in the range of from 1/0.5/0.2 to 1/1.9/1.5, preferably in the range of from 1/0.7/0.4 to 1/1.5/1.1.

The index n represents 3 to 50, preferably 3 to 30, in particular 3 to 25 and specifically 3 to 15. In a further preferred embodiment, n represents at least 10 to 50, preferably 10.3 to 30, particularly preferably 10.5 to 25 and specifically 11 to 20.

Suitable X radicals are NH or O, preferably O. In a further preferred embodiment, X is NH or O and R¹ is —[CH₂CH₂NH]ₙ—H.

R² is usually H or CI, preferably H.

R³ is usually H or C₁-C₈-alkyl, preferably C₁-C₈-alkyl, more preferably branched C₁-C₈-alkyl, and particularly tert-butyl.

In one particularly preferred embodiment, the UV absorber conforms to the formula II

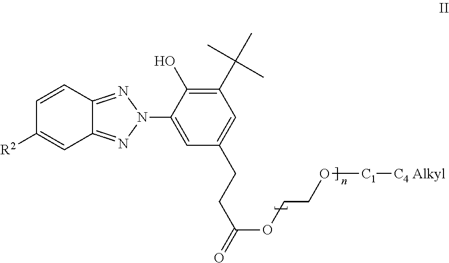

where R² and n are each as defined above. The expression “C₁-C₄ alkyl” typically represents a linear or branched, preferably linear, alkyl group. It preferably comprises 1 to 4, more preferably 1 to 2 and particularly one carbon atom.

The UV absorbers conventionally have a surface tension (ST) at the interface of water to air at 25° C. of at most 50 mN/m, preferably of at most 46 mN/m, particularly preferably at most 44 mN/m, specifically at most 40 mN/m. The ST is typically at least 25 mN/m, preferably at least 30 mN/m.

The agrochemical composition according to the invention in general comprises 0.1 to 50 wt. %, preferably 0.5 to 30 wt. %, particularly preferably 1.0 to 15 wt. % of UV absorber, in each case based on the composition.

The term pesticide designates at least one active compound chosen from the group of fungicides, insecticides, nematicides, herbicides, safeners and/or growth regulators. Preferred pesticides are fungicides, insecticides and herbicides, in particular insecticides. Mixtures of pesticides from two or more of the abovementioned classes can also be used. The person skilled in the art is familiar with such pesticides, which can be found, for example, in Pesticide Manual, 14th Ed. (2006), The British Crop Protection Council, London. Suitable insecticides are insecticides from the class of carbamates, organophosphates, organochlorine insecticides, phenylpyrazoles, pyrethroids, neonicotinoids, spinosins, avermectins, milbemycins, juvenile hormone analogues, alkyl halides, organotin compounds, nereistoxin analogues, benzoylureas, diacylhydrazines, METI acaricides, and insecticides such as chloropicrin, pymetrozine, flonicamid, clofentezine, hexythiazox, etoxazole, diafenthiuron, propargite, tetradifon, chlorfenapyr, DNOC, buprofezin, cyromazine, amitraz, hydramethylnon, acequinocyl, fluacrypyrim, rotenone, or derivatives thereof. Suitable fungicides are fungicides of the classes dinitroanilines, allylamines, anilinopyrimidines, antibiotics, aromatic hydrocarbons, benzenesulfonamides, benzimidazoles, benzisothiazoles, benzophenones, benzothiadiazoles, benzotriazines, benzylcarbamates, carbamates, carboxamides, carboxylic acid amides, chloronitriles, cyanoacetamide oximes, cyanoimidazoles, cyclopropanecarboxamides, dicarboximides, dihydrodioxazines, dinitrophenylcrotonates, dithiocarbamates, dithiolanes, ethylphosphonates, ethylaminothiazolecarboxamides, guanidines, hydroxy-(2-amino-)pyrimidines, hydroxyanilides, imidazoles, imidazolinones, inorganic compounds, isobenzofuranones, methoxyacrylates, methoxycarbamates, morpholines, N-phenylcarbamates, oxazolidinediones, oximinoacetates, oximinoacetamides, peptidylpyrimidine nucleosides, phenylacetamides, phenylamides, phenylpyrroles, phenylureas, phosphonates, phosphorothiolates, phthalamic acids, phthalimides, piperazines, piperidines, propionamides, pyridazinones, pyridines, pyridinylmethylbenzamides, pyrimidinamines, pyrimidines, pyrimidinonehydrazones, pyrroloquinolinones, quinazolinones, quinolines, quinones, sulfamides, sulfamoyltriazoles, thiazolecarboxamides, thiocarbamates, thiocarbamates, thiophanates, thiophenecarboxamides, toluamides, triphenyltin compounds, triazines, triazoles. Suitable herbicides are herbicides of the classes of acetamides, amides, aryloxyphenoxypropionates, benzamides, benzofuran, benzoic acids, benzothiadiazinones, bipyridylium, carbamates, chloroacetamides, chlorocarboxylic acids, cyclohexanediones, dinitroanilines, dinitrophenol, diphenyl ethers, glycines, imidazolinones, isoxazoles, isoxazolidinones, nitriles, N-phenylphthalimides, oxadiazoles, oxazolidinediones, oxyacetamides, phenoxycarboxylic acids, phenylcarbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl(thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolinones, triazolocarboxamides, triazolopyrimidines, triketones, uracils, ureas.

Preferred herbicides are napropamide, propanil, bentazone, paraquat dichloride, cycloxydim, sethoxydim, ethalfluralin, oryzalin, pendimethalin, trifluralin, acifluren, aclonifen, fomesafen, oxyfluoren, ioxynil, imazetapyr, imazaquin, chloridazon, norflurazon, thiazopyr, triclopyr, dithiopyr, diflufenican, picolinafen, amidosulfuron, molinate, vernolate, prometon, metribuzin, azafenidin, carfentrazone-ethyl, sulfentrazone, metoxuron, monolinuron, fluchloralin and flurenol. Preferred fungicides are cyprodinil, fuberidazole, dimethomorph, procloraz, triflumizole, tridemorph, edifenfos, fenarimol, nuarimol, ethirimol, quinoxylen, dithianon, metominostrobin, trifloxystrobin, dichlofluamid, bromuconazole and myclobutanil. Preferred insecticides are acephate, azinphos-ethyl, azinphos-methyl, isofenphos, chlorpyriphos-methyl, dimethylvinphos, phorate, phoxim, prothiofos, cyhexatin, alanycarb, ethiofencarb, pirimicarb, thiodicarb, fipronil, bioallethrin, bioresmethin, deltamethrin, fenpropathin, flucythrinate, taufluvalinate, alpha-cypermethrin, metaflumizone, zeta-cypermethrin, resmethin, tefluthrin, lambda cyhalothrin and hydramethylnon. In a further embodiment, preferred pesticides are pyrethroids or metaflumizone, in particular pyrethroids. Particularly preferred pesticides are alpha-cypermethrin and metaflumizone.

In one embodiment, pesticides which are UV-sensitive are used. This UV sensitivity can be determined in simple preliminary tests. Pesticides are preferably regarded as UV-sensitive if on irradiation with UVNIS light of wavelength 300-800 nm, of a pesticide film which by drying of a 25 wt. % solution of the pesticide in a suitable solvent, preferably in acetone, they are degraded to the extent of at least 20 wt. % within 24 h at 25° C. The illuminance used is typically in the range from 10 000 to 100 000 lux, preferably in the range from 50 000 to 80 000 lux. The pesticides her are regarded as degraded if the concentration of the pesticide (or one pesticidal component in a mixture of a plurality of pesticidal components) is reduced correspondingly.

The agrochemical composition according to the invention in general comprises 0.01 to 95 wt. %, preferably 0.5 to 80 wt. %, particularly preferably 2 to 50 wt. % and specifically 5 to 20 wt. % of pesticide, in each case based on the composition.

The weight ratio of pesticide to UV absorber is usually from 30:1 to 1:3, preferably 15:1 to 1:2, particularly preferably 8:1 to 1:1.

Agrochemical composition comprising pesticide and UV absorber can be in composition types conventional for agrochemical formulations, e.g. solutions, emulsions, suspensions dusts, powders, pastes and granules. The type depends on the particular intended use; it should in all cases ensure a fine and uniform distribution of the compound according to the invention. Examples of composition types are suspensions (SC, OD, FS), emulsifiable concentrates (EC), emulsions (EW, EO, ES), pastes, pastilles, wettable powders or dusts (WP, SP, SS, WS, DP, DS) or granules (GR, FG, GG, MG), which are either soluble or dispersible in water, and gels for treatment of plant propagation materials, such as seed (GF). Baits for animals, such as ants or rats, can be in various of the abovementioned composition types, preferably as powders, pastes, granules or gels. In general the compositions types (e.g. SC, EC, OD, FS, WG, SG, WP, SP, SS, WS, GF) are employed in diluted form. Composition types such as DP, DS, GR, FG, GG, MG or baits are as a rule employed in undiluted form. Preferred composition types are suspensions.

The agrochemical compositions can furthermore also comprise auxiliaries conventional for plant protection compositions, the choice of the auxiliaries depending on the concrete use form or the active compound. Examples of suitable auxiliaries are solvents, solid carrier substances, surface-active substances (such as further solubilizing agents, protective colloids, wetting agents and adhesive agents), organic and inorganic thickeners, bactericides, antifreeze agents, defoamers, optionally dyestuffs and adhesives (e.g. for seed treatment) or conventional auxiliaries for bait formulation (e.g. attractants, feedstuffs, bitters).

Possible solvents are water, organic solvents, such as mineral oil fractions of medium to high boiling point, such as kerosene and diesel oil, furthermore coal tar oils and oils of plant or animal origin, aliphatic, cyclic and aromatic hydrocarbons, e.g. paraffins, tetrahydronaphthalene, alkylated naphthalenes and derivatives thereof, alkylated benzenes and derivatives thereof, alcohols, such as methanol, ethanol, propanol, butanol and cyclohexanol, glycols, ketones, such as cyclohexanone, gamma-butyrolactone, dimethyl-fatty acid amides, fatty acids and fatty acid esters and strongly polar solvents, e.g. amines, such as N-methylpyrrolidone. In principle, solvent mixtures can also be used, as well as mixture of the abovementioned solvents and water.

Solid carrier substances are mineral earths, such as silicas, silica gels, silicates, talc, kaolin, limestone, lime, chalk, bolus, loam, clay, dolomite, diatomaceous earth, calcium sulfate and magnesium sulfate, magnesium oxide, ground plastics, fertilizers, such as ammonium sulfate, ammonium phosphate, ammonium nitrate, ureas and plant products, such as cereal flour, tree bark, wood and nutshell flour, cellulose powder or other solid carrier substances.

Possible surface-active substances (adjuvants, wetting, adhesive, dispersing or emulsifying agents) are the alkali metal, alkaline earth metal and ammonium salts of aromatic sulfonic acids, e.g. of lignin—(Borresperse® types Borregaard, Norway), phenol-, naphthalene—(Morwet® types, Akzo Nobel, USA) and dibutylnaphthalenesulfonic acid (Nekal® types, BASF, Germany), and of fatty acids, alkyl- and alkylarylsulfonates, alkyl, lauryl ether and fatty alcohol sulfates, and salts of sulfated hexa-, hepta- and octadecanols, as well as of fatty alcohol glycol ethers, condensation products of sulfonated naphthalene and its derivatives with formaldehyde, condensation products of naphthalene or of naphthalenesulfonic acids with phenol and formaldehyde, polyoxyethylene octylphenol ethers, ethoxylated isooctyl-, octyl- or nonylphenol, alkylphenyl or tributylphenyl polyglycol ethers, alkylaryl polyether alcohols, isotridecyl alcohol, fatty alcohol-ethylene oxide condensates, ethoxylated castor oil, polyoxyethylene or polyoxypropylene alkyl ethers, lauryl alcohol polyglycol ether acetate, sorbitol esters, lignin-sulfite waste liquors and proteins, denatured proteins, polysaccharides (e.g. methylcellulose), hydrophobically modified starches, polyvinyl alcohol (Mowiol® types, Clariant, Switzerland), polycarboxylates (Sokalan® types, BASF, Germany), polyalkoxylates, polyvinylamine (Lupamin® types, BASF, Germany), polyethyleneimine (Lupasol® types, BASF, Germany), polyvinylpyrrolidone and copolymers thereof.

Examples of thickeners (i.e. compounds which impart to the composition modified flow properties, i.e. high viscosity in the resting state and low viscosity in the agitated state) are polysaccharides and organic and inorganic laminar minerals, such as xanthan gum (Keizan®, CP Kelco, USA), Rhodopol® 23 (Rhodia, France) or Veegum® (R.T. Vanderbilt, USA) or Attaclay® (Engelhard Corp., NJ, USA).

Bactericides can be added to stabilize the composition. Examples of bactericides are those based on dichlorophen and benzyl alcohol hemiformal and isothiazolinone derivatives, such as alkylisothiazolinones and benzisothiazolinones (Acticide® MBS from Thor Chemie).

Examples of suitable antifreeze agents are ethylene glycol, propylene glycol, urea and glycerol.

Examples of defoamers are silicone emulsions (such as e.g. Silikon® SRE, Wacker, Germany or Rhodorsil®, Rhodia, France), long-chain alcohols, fatty acids, salts of fatty acids, organofluorine compounds and mixtures thereof.

Examples of composition types are:

1. Composition types for dilution in water
i) Water-Soluble Concentrates (SL, LS)
   10 parts by wt. of the pesticide are dissolved with 90 parts by wt. of water or a water-soluble solvent. Alternatively, wetting agents or other auxiliaries are added. During the dilution in water, the pesticide dissolves. A composition with an active compound content of 10 wt. % is obtained in this manner.
ii) Dispersible Concentrates (DC)
   20 parts by wt. of the pesticide are dissolved in 70 parts by wt. of cyclohexanone with the addition of 10 parts by wt. of a dispersing agent, e.g. polyvinylpyrrolidone. On dilution in water, a dispersion results. The active compound content is 20 wt. %
iii) Emulsifiable Concentrates (EC)
   15 parts by wt. of the pesticide are dissolved in 75 parts by wt. of xylene with the addition of Ca dodecylbenzenesulfonate and castor oil ethoxylate (in each case 5 parts by wt.). On dilution in water, an emulsion results. The composition has an active compound content of 15 wt. %.
iv) Emulsions (EW, EO, ES)
   25 parts by wt. of the pesticide are dissolved in 35 parts by wt. of xylene with the addition of Ca dodecylbenzenesulfonate and castor oil ethoxylate (in each case 5 parts by wt.). This mixture is added to 30 parts by wt. of water and converted into a homogeneous emulsion by means of an emulsifying machine (e.g. Ultra-Turrax). On dilution in water, an emulsion results. The composition has an active compound content of 25 wt. %.
v) Suspensions (SC, OD, FS)
   20 parts by wt. of the pesticide are comminuted in a stirred ball mill with the addition of 10 parts by wt. of dispersing and wetting agents and 70 parts by wt. of water or an organic solvent to give a fine suspension of the active compound. On dilution in water, a stable suspension of the active compound results. The active compound content in the composition is 20 wt. %
vi) Water-Dispersible and Water-Soluble Granules (WG, SG)
   50 parts by wt. of the pesticide are finely ground with the addition of 50 parts by wt. of dispersing and wetting agents and are prepared as water-dispersible or water-soluble granules by means of industrial equipment (e.g. extrusion, spray tower, fluidized bed). On dilution in water, a stable dispersion or solution of the active compound results. The composition has an active compound content of 50 wt. %.
vii) Water-Dispersible and Water-Soluble Powders (WP, SP, SS, WS)
   75 parts by wt. of the pesticide are ground in a rotor-stator mill with the addition of 25 parts by wt. of dispersing and wetting agents and silica gel. On dilution in water, a stable dispersion or solution of the active compound results. The active compound content of the composition is 75 wt. %
viii) Gels (GF)
   20 parts by wt. of the pesticide, 10 parts by wt. of dispersing agent, 1 part by wt. of swelling agent (“gelling agent”) and 70 parts by wt. of water or an organic solvent are ground in a ball mill to give a fine suspension. On dilution with water, a stable suspension with an active compound content of 20 wt. % results.

2. Composition Types for Direct Application
ix) Dusts (DP, DS)
   5 parts by wt. of the pesticide are finely ground and mixed intimately with 95 parts by wt. of finely divided kaolin. A dust composition with an active compound content of 5 wt. % is thereby obtained.
x) Granules (GR, FG, GG, MG)
   0.5 part by wt. of the pesticide are finely ground and bound with 99.5 parts by wt. of carrier substances. The usual processes here are extrusion, spray drying or fluidized bed. Granules for direct application with an active compound content of 0.5 wt. % are thereby obtained.
xi) ULV Solution (UL)
   10 parts by wt. of the pesticide are dissolved with 90 parts by wt. of an organic solvent, e.g. xylene. A composition for direct application with an active compound content of 10 wt. % are thereby obtained.

The compounds can be used as such or in the form of their compositions, e.g. in the form of directly sprayable solutions, powders, suspensions, dispersions, emulsions, oil dispersions, pastes, dust compositions, scattering compositions or granules, by spraying, misting, dusting, scattering, laying out of baits, brushing, dipping or pouring. Aqueous use forms can be prepared from emulsion concentrates, pastes or wettable powders (spray powders, oil dispersions) by addition of water. For the preparation of emulsions, pastes or oil dispersions, the substances can be homogenized in water as such or as a solution in an oil or solvent, by means of wetting, adhesive, dispersing or emulsifying agents. However, concentrates comprising wetting, adhesive, dispersing or emulsifying agent and possibly solvent or oil which are suitable for dilution with water can also be prepared from the active substance.

The active compound concentrations in the ready-to-use formulations can be varied within relatively wide ranges. In general, they are between 0.0001 and 10%, preferably between 0.01 and 1%. The amounts applied for use in plant protection are between 0.01 and 2.0 kg of active compound per ha, depending on the nature of the desired effect. In the treatment of plant propagation materials, e.g. seed, in general active compound amounts of from 1 to 1,000 g/100 kg, preferably 5 to 100 g/100 kg of propagation material or seed are used. For use in the protection of material or stored products, the amount of active compound applied depends on the nature of the field of use and of the desired effect. Conventional amounts applied in the protection of materials are, for example, 0.001 g to 2 kg, preferably 0.005 to 1 kg of active compound per cubic meter of material treated.

The present invention furthermore relates to UV absorbers of the formula III

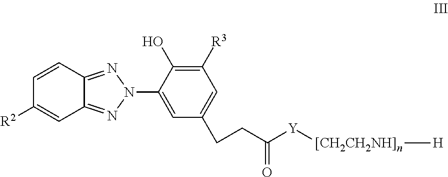

wherein Y represents NH or O and R², R³, n, m and the expression “[CH₂CH₂NH]ₙ—H” are as defined above. In a preferred embodiment, Y represents NH.

The UV absorbers of the formula III according to the invention conventionally have a surface tension at the interface of water to air at 25° C. of at most 50 mN/m, preferably of at most 46 mN/m, particularly preferably at most 44 mN/m, specifically at most 40 mN/m. The ST is typically at least 25 mN/m, preferably at least 30 mN/m.

The present invention furthermore relates to a use of the UV absorbers of the formula III according to the invention in agrochemical compositions. Use in agrochemical compositions is preferred. Use to stabilize UV-sensitive pesticides, especially against sunlight, is particularly preferred. Suitable agrochemical compositions are as described above.

The present invention furthermore relates to a method of controlling phytopathogenic fungi and/or undesirable plant growth and/or undesirable insect or mite infestation and/or of regulating plant growth, wherein the composition according to the invention is allowed to act on the particular pests, their habitat or the plants to be protected from the particular pest, the soil and/or on the undesirable plants and/or the crop plants and/or their habitat.

An advantage of the present invention is that they stabilize UV-sensitive pesticides, in particular already at low concentrations of UV absorber. A further advantage is that fewer surfactants need to be employed in order to obtain a high stability of active compound dispersions, in particular active compound suspensions. It is also of advantage that the UV absorbers of the present invention are capable of reducing the surface tension of water to a greater extent, i.e., that they have a higher surface activity. The UV absorbers are readily soluble in agrochemical formulations, or very readily compatible with agrochemical formulations, such as aqueous emulsions and aqueous suspensions. For example, also no additional emulsifier is necessary in order to incorporate the UV absorbers into the formulation.

The following examples illustrate the invention without limiting it.

### EXAMPLES

Pluriol® A350E: Polyalkoxylene glycol monomethyl ether, OH number approx. 160 mg of KOH/g, molar mass approx. 350 g/mol, determined by means of OH number, commercially obtainable as Pluriol® A350E from BASF.
Pluriol® A500E: Methyl-polyethylene glycol, OH number approx. 110 mg of KOH/g, molar mass approx. 500 g/mol, determined by means of OH number, commercially obtainable as Pluriol® A500E from BASF SE.
Pluronic® 10500: Poly(ethylene glycol-block-propylene-block-ethylene glycol), with a propylene glycol block of a molar mass of 3,250 g/mol and a total molecular weight of 6,500 g/mol (commercially obtainable as Pluronic® PE 10500 from BASF SE).
Bactericide: Aqueous mixture of 2.5 wt. % of 2-methyl-4-isothiazolin-3-one (MIT) and 2.5 wt. % of 1,2-benzisothiazolin-3-one (BIT) (commercially obtainable as Acticide® MBS from Thor)
Defoamer: Silicone-based, active content 20 wt. % (commercially obtainable as Silikon SRE-PFL from Wacker)
Xanthan: Granular xanthan gum, 14 wt. % water content, viscosity 2,000 mPas as a 0.3 wt. % solution by the Brookfield method (commercially obtainable as Rhodopol® G from Rhodia).
Alverde®: Water-based suspension concentrate of metaflumizone, comprising 1.8-2.5 wt. % of sodium dioctylsulfate, 3.1-3.9 wt. % of ethoxylated isodecyl alcohol and 2.7-3.3 wt. % of polyarylphenol ethoxylate, commercially available from BASF SE.
Lupasol® FG: A polyethyleneimine having an average molar mass of about 800 g/mol (determined by means of GPC), a ratio of primary to secondary to tertiary amine groups of about 1 to 0.9 to 0.5 and a water content of about 1 wt. %, commercially obtainable as Lupasol® FG from BASF SE.
Tinuvin® 384-2: A UV absorber commercially available from CIBA AG from the class of the hydroxyphenylbenzotriazoles (95% benzenepropanoic acid 3-(2H-benzotriazol-2-yl)-5-(1,1-dimethylethyl)-4-hydroxy-C7-9-alkyl ester and 5% 1-methoxy-2-propyl acetate).
Tinuvin® 109: A UV absorber commercially available from CIBA AG from the class of the hydroxyphenyltriazoles (mixture of 45-55 wt. % of octyl 3-(5-chloro-2H-benzotriazol-2-yl)-5-(1,1-dimethylethyl)-4-hydroxyphenylpropanoate and 45-55 wt. % of 2-ethylhexyl 3-(5-chloro-2H-benzotriazol-2-yl)-5-(1,1-dimethylphenyl)-4-hydroxyphenylpropanoate).
Uvinul® P25: p-Aminobenzoic acid ethoxylate (45) (molecular weight approx. 1,265 g/mol, the sum of x+y+z is about 25) is a commercially obtainable product with the name Uvinul® P25 from BASF SE.

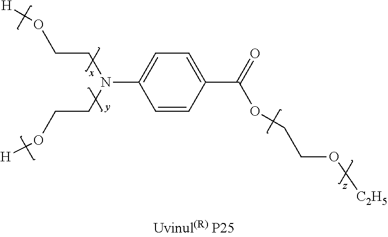

### Example 1A

### Synthesis of 3-(3-benzotriazol-2-yl-5-tert-butyl-4-hydroxyphenyl)propionic acid (2)

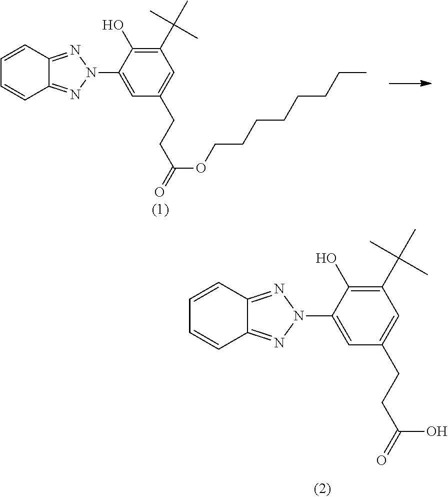

361 g (0.8 mol) (1, Tinuvin® 384-2) were suspended in 1.28 L of 75% ethanol and then admixed with 128 g (1.6 mol) of 50% strength aqueous sodium hydroxide solution. The mixture was stirred at 20° C. overnight. Thereafter, the solution was admixed with 183 g (1.6 mol) of 32% strength hydrochloric acid. The suspension was filtered off with suction and washed 3 times with 2 L of water each time. When dried, 251 g of a white powder (2) were obtained (92% yield).

### Example 1B

### Synthesis of methyl 3-(3-benzotriazol-2-yl-5-tert-butyl-4-hydroxyphenyl)propionate (3)

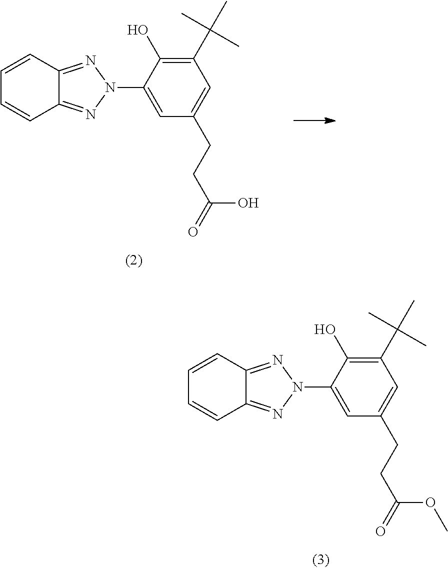

To a suspension of 70 g (0.21 mol) of (2) in 1.6 L of methanol were added 5 ml of 98% strength sulfuric acid followed by stirring under reflux for 6 hours. The suspension was filtered at 20° C. and the product was washed 2 times with 350 ml of methanol each time. When dried, 69 g of a white powder (3) were obtained (yield=93%).

### Example 1C

### Synthesis of 3-(3-benzotriazol-2-yl-5-tert-butyl-4-hydroxyphenyl)propionic acid [Pluriol A350E]ester (5)

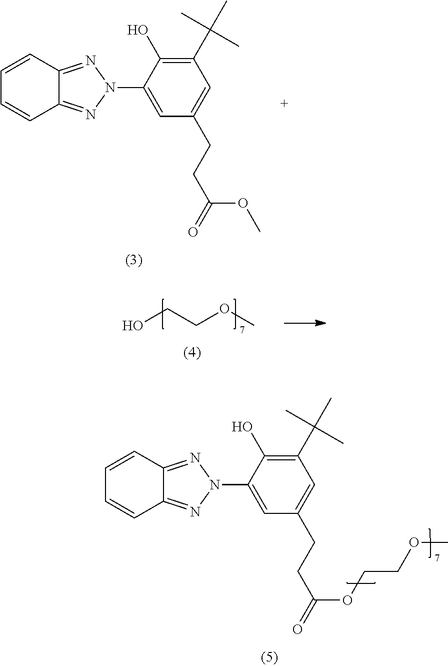

Nitrogen was passed through by immersion during the entire reaction time. 59.7 g (187 mmol) of Pluriol® A350E (4) were stirred at 150° C. for 30 minutes. Then, 60.0 g (170 mmol) (3) and 0.54 g (2 mmol) of titanium(IV) isopropoxide were added. The reaction mixture was stirred at 155° C. for 18 hours. The methanol formed was distilled off. The mixture was taken up in 100 ml of toluene and 350 mg (3 mmol) of 85% strength phosphoric acid were added, and the solution was left to stand at 20° C. for 24 hours. The product was purified by flash chromatography on silica gel 60. For this, the solution was introduced onto the flash column and the educt (3) was eluted from the column with toluene followed by the product (5) with methanol. The methanolic solution was concentrated to dryness on a rotary evaporator to obtain 106 g of reddish orange viscous liquid (6) (yield=99%).

### Example 1D

### Synthesis of 3-(3-benzotriazol-2-yl-5-tert-butyl-4-hydroxyphenyl)propionic acid [Pluriol A500E]ester (7)

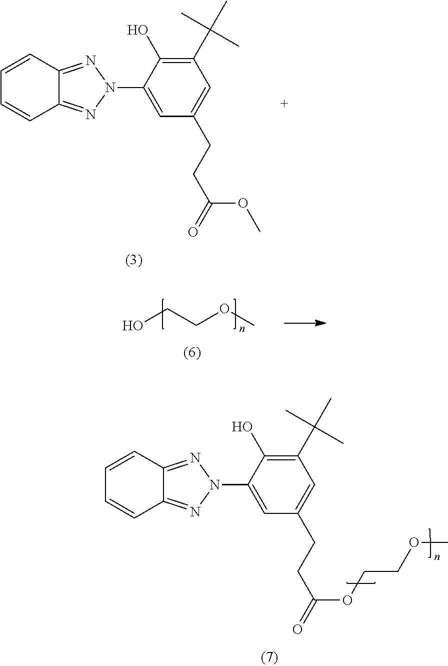

Nitrogen was passed through by immersion during the entire reaction time. 28.8 g (62.3 mmol) of Pluriol® A500E (6) and 20.0 g (56.6 mmol) of (3) were initially charged at 20° C. and stirred at 155° C. for 30 minutes. Then, 0.54 g (1.9 mmol) of titanium(IV) isopropoxide was added and the reaction mixture was stirred at 155° C. for 52 hours. The methanol formed was distilled off. The mixture was taken up in 100 ml of toluene. The product was purified by flash chromatography with 430 g of silica gel 60. For this, the solution was introduced onto the flash column and the educt (3) was eluted from the column with a mixture of toluene and ethyl acetate (15:2% by volume) followed by the product (7) with methanol. The methanolic solution was concentrated to dryness on a rotary evaporator to obtain 43 g of reddish orange viscous liquid (7) (yield=97%).

### Example 1E

### Synthesis of 3-(3-benzotriazol-2-yl-tert-butyl-4-hydroxyphenyl)propionic acid [tridecanol-EO₂₄]ester (9)

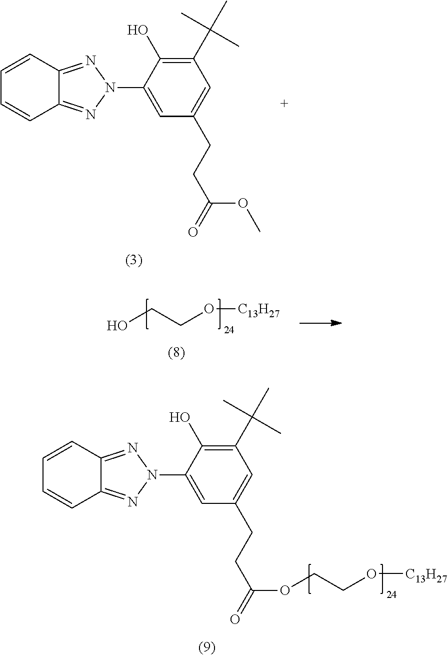

Nitrogen was passed through by immersion during the entire reaction time. 8.84 g (25 mmol) of (3) and 31.17 g (27.5 mmol) of (8) were stirred at 155° C. for 30 minutes. Then, 0.07 g (0.25 mmol) of titanium(IV) isopropoxide was added. The reaction mixture was stirred at 155° C. for 5 hours. A further 0.07 g (0.25 mmol) of titanium(IV) isopropoxide was added followed by stirring at 155° C. for a further 17 hours. The methanol formed was distilled off. The product was purified by column chromatography with 0.15 kg of silica gel 60. For this, the solution was introduced onto the flash column and the educt (3) was eluted from the column with ethyl acetate followed by the product (9) with methanol. The methanolic solution was concentrated to dryness on a rotary evaporator to obtain 15.2 g of yellow viscous liquid (12) (yield=38.5%).

### Example 1F

### Synthesis of 3-(3-benzotriazol-2-yl-5-tert-butyl-4-hydroxyphenyl)propionic acid [tridecanol-EO₁₈]ester (11)

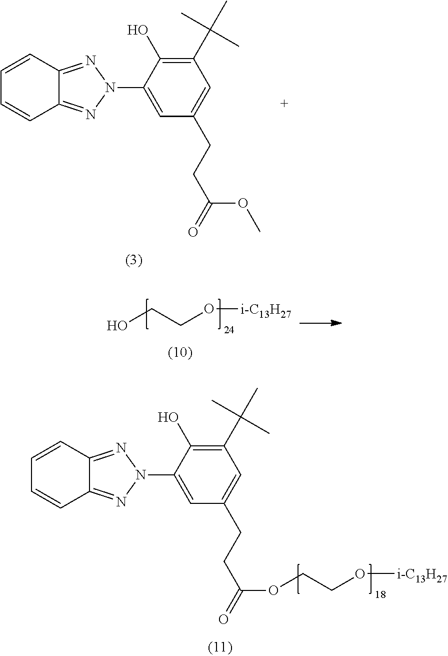

Nitrogen was passed through by immersion during the entire reaction time. 8.84 g (25 mmol) of (3) and 23.55 g (27.5 mmol) of (10) were stirred at 155° C. for 30 minutes. Then, 0.07 g (0.25 mmol) of titanium(IV) isopropoxide was added. The reaction mixture was stirred at 155° C. for 5 hours. A further 0.07 g (0.25 mmol) of titanium(IV) isopropoxide was added followed by stirring at 155° C. for a further 17 hours. The methanol formed was distilled off. The product was purified by column chromatography on silica gel. For this, the solution was introduced onto the flash column and the educt (3) was eluted from the column with ethyl acetate followed by the product (10) with methanol. The methanolic solution was concentrated to dryness on a rotary evaporator to obtain 25 g of yellow viscous liquid (11) (yield=76.1%).

### Example 1G

### Synthesis of 2-(5-chloro-2H-benzotriazol-2-yl)-6-tert-butyl-4-hydroxyphenylpropionic acid (13)

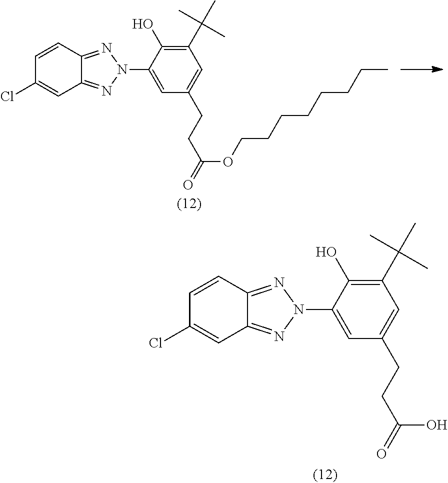

389 g (0.8 mol) of Tinuvin® 109 (12) were suspended in 0.96 l of 75% strength ethanol and then admixed with 128 g (1.6 mol) of 50% strength aqueous sodium hydroxide solution. The mixture was stirred at 20° C. overnight. It was subsequently diluted with 6 l of water and mixed with 183 g (1.6 mol) of 32% strength hydrochloric acid. The suspension was filtered off with suction and the solid was washed 3 times with 2 l of water each time. When dried, 291 g (13) were obtained as a white powder (97% yield).

### Example 1H

### Synthesis of 2-(5-chloro-2H-benzotriazol-2-yl)-6-tert-butyl-4-hydroxyphenylpropionic acid methyl ester (14)

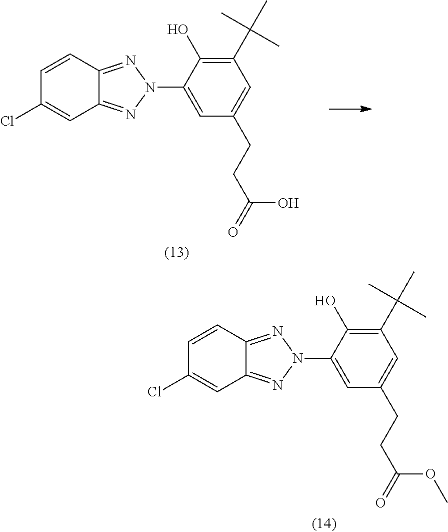

To a suspension of 37.4 g (0.1 mol) of (13) in 0.75 l of methanol were added 5 ml of 98% strength sulfuric acid followed by stirring under reflux for 4 hours. The suspension was filtered and the product was washed with methanol to obtain, when dried, 36 g of a white powder (14) (yield=93%).

### Example 1J

### Synthesis of 2-(5-chloro-2H-benzotriazol-2-yl)-6-tert-butyl-4-hydroxyphenylpropionic acid [Pluriol A350E]ester (15)

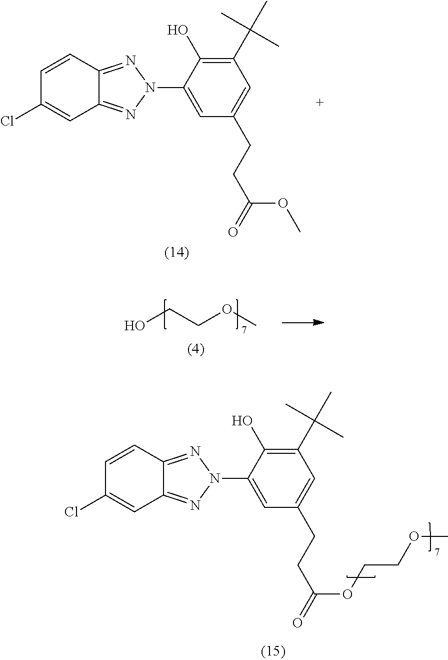

Nitrogen was passed through by immersion during the entire reaction time. 33 g (103 mmol) of Pluriol A350E (4) were stirred at 150° C. for 30 minutes. Then, 36.4 g (93.8 mmol) (14) and 0.3 g (1 mmol) of titanium(IV) isopropoxide were added. The reaction mixture was stirred at 155° C. for 21 hours. The methanol formed was distilled off. The mixture was taken up in 250 ml of methylene chloride and 173 mg (1.5 mmol) of 85% strength phosphoric acid were added, and the solution was left to stand at 20° C. for 24 hours. The product was purified by flash chromatography on silica gel 60. For this, the solution was introduced onto the flash column and the educt (14) was eluted from the column with methylene chloride followed by the product (15) with methanol. The methanolic solution was concentrated to dryness on a rotary evaporator to obtain 52 g of red viscous liquid (15) (yield=83%).

### Example 1K

### Synthesis of 3-(3-benzotriazol-2-yl-5-tert-butyl-4-hydroxyphenyl)-propionic acid Lupasol FG amide (17)

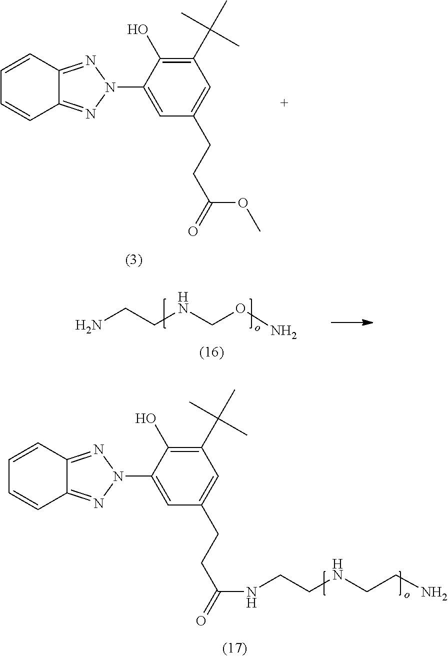

Nitrogen was passed through by immersion during the entire reaction time. 21.7 g (61.3 mmol; 5 mol %) of (3) and 75 g (1230 mol) of Lupasol FG (16) (amine number 16.34 mmol/g) were mixed at 90° C. and stirred at 110° C. for 5 hours. The ethanol formed was distilled off to obtain 91 g of an orange viscous liquid (17) (yield=96%).

### Example 1L

### Synthesis of 3-(3-benzotriazol-2-yl-5-tert-butyl-4-hydroxyphenyl)-propionic acid Lupasol FG amide (18)

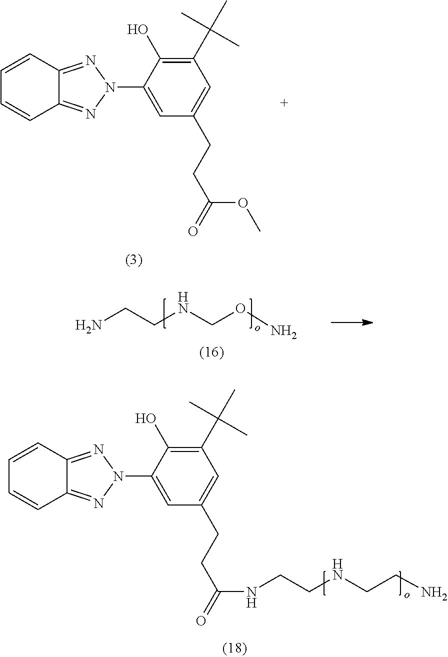

Nitrogen was passed through by immersion during the entire reaction time. 31.8 g (90 mmol; 10 mol %) of (3) and 55 g (900 mmol) of Lupasol FG (16) were mixed at 90° C. and stirred at 110° C. for 6 hours. The ethanol formed was distilled off to obtain 78 g of a highly viscous orange liquid (18) (yield=93%).

### Example 2

### Stability of Suspension Concentrate

The suspension concentrates A18 and A19 were prepared by wet grinding (1 h, at 3000 rpm, Dispermat) of 105 g of metaflumizone (95% by weight purity), 70 g of 1,2-propylene glycol and Pluronic® 10500 (amount see Table 1). To obtain a homogeneous, stable suspension in which at least 90% of the solid particles had a particle size of less than 5 μm and D(4,3) was 1.0 μm. These suspensions were each admixed with 40 g of UV absorber 5 or 7 (see Table 31 except for experiment C1), 20 g of Wetol D1, 5.0 g of defoamer, 3.0 g of xanthan and 2.0 g of bactericide with vigorous stirring and made up to 1.0 l with water.

After storage at 54° C. for two weeks, no change in the particle size and the particle distribution of the active ingredient was found.

A sample from all the experiments was in each case diluted with CIPAC water D (containing 342 ppm of Ca/Mg ions) to a 0.1 and 1 wt. % strength suspension of the active ingredient. Practically no sediments of the active ingredient were found after storage for 6 h.

TABLE 1

| | | Amount of Pluronic ® |
|---|---|---|
| | UV absorber | 10500 added |
| Experiment | from Example | [g/l] |
| C1^{a)} | none | 167 |
| C2^{a)} | none | 111 |
| A18 | 1C | 111 |
| A19 | 1D | 111 |

^{a)}not according to the invention

The experiment shows that the addition of UV absorbers according to the present invention does not decrease the stability of suspension concentrates. The high stability was again achieved at a low concentration of Pluronic® 10500 dispersing agent.

### Example 3

### Stability of Suspension Concentrate

The suspension concentrates A32 and A33 were prepared as in Example 2, except that the concentration of Pluronic 10500 was 222 g/l and that of UV absorber was 80 g/l (Table 2).

After storage at 54° C. for two weeks, no change in the particle size and the particle distribution was found.

A sample from all the experiments was in each case diluted with CIPAC water D (comprising 342 ppm of Ca/Mg ions) to a 0.1 and 1 wt. % strength suspension. Practically no sediments were found after storage for 6 h.

TABLE 2

| | UV absorber | Amount of Pluronic ® |
|---|---|---|
| | from | 10500 added |
| Experiment | Example | [g/l] |
| C3^{a)} | none | 222 |
| C4^{a)} | Uvinul ® P25 | 222 |
| A32 | 1C | 222 |
| A33 | 1D | 222 |

^{a)}not according to the invention

The experiment shows that the addition of UV absorbers according to the invention does not decrease the stability of the suspension concentration.

### Example 4A

### UV-Stabilizing Effect A formulation comprising 100 g/l of metaflumizone and 40 g/l of UV absorber was prepared as described in Example 2. The formulation was diluted with water such that an active content of 10 g/l (Table 3) or 1 g/l (Table 4) was obtained.

### Analysis:

About 20 mg of the formulation thus prepared were placed on a microscope slide, allowed to dry for 30 minutes and then exposed to light for one or seven days continuously (Atlas Suntest CRT plus, “Outdoor” setting, irradiation corresponds to the same spectrum and intensity as normal sunlight at midday in summer). After the exposure time, the samples were dissolved in dimethyl sulfoxide. The residual level of metaflumizone was determined by means of quantitative HPLC (column: BEH C18 1.7 μm 2.1×100; elution with a gradient of acetonitrile/0.1% H3PO4 rising from 5/95:95/5). For comparison, each exposure series run included a sample without UV absorber (reference). Evaluation: 2 samples were treated as blank samples by immediately redissolving them after drying, without exposure to light, and determining the metaflumizone content. The resulting mean was set equal to 100% and the remaining samples were standardized against that. The results are summarized in Tables 3 and 4.

TABLE 3
(10 g/l metaflumizone)

| Structure of UV | | |
|---|---|---|
| absorber of | Residual metaflumizone | Residual metaflumizone |
| Example | content (%) after 1 day | content (%) after 1 week |
| No UV absorber^{a)} | 31 | 7 |
| 1C | 89 | 55 |
| Tinuvin 1130^{a)} | 78 | 27 |

^{a)}not according to the invention

TABLE 4
(1 g/l metaflumizone)

| Structure of UV | | |
|---|---|---|
| absorber of | Residual metaflumizone | Residual metaflumizone |
| Example | content (%) after 1 day | content (%) after 1 week |
| No UV Absorber^{a)} | 12 | 0 |
| 1C | 45 | 16 |
| Tinuvin 1130^{a)} | 40 | 6 |

^{a)}not according to the invention

### Example 4B

### UV-Stabilizing Effect

A commercial formulation of metaflumizone (Alverde®) comprising 240 g/l of metaflumizone was diluted with water to an active content of 10 g/l. Various UV absorbers were added (Table 5) and analyzed as described under Example 4A. The results are summarized in Table 5.

TABLE 5

| Structure of UV | | |
|---|---|---|
| absorber of | Residual metaflumizone | Residual metaflumizone |
| Example | content (%) after 1 day | content (%) after 1 week |
| No UV Absorber^{a)} | 3 | 0 |
| 1C | 34 | 1 |
| 1I | 29 | 11 |
| 1J | 46 | 30 |

^{a)}not according to the invention

The data from Examples 4A and 4B showed that less pesticide is degraded with the UV absorbers according to the present invention than without UV absorbers. It was also shown that less pesticide is degraded in the presence of the UV absorbers according to the present invention than in the presence of Tinuvin 1130.

### Example 3

### Biological Tests

The plants (lima beans, Phaseolus lunatus) were irradiated with UV light constantly for 24 h per day at 26° C. in a growth chamber. The irradiation intensity in the UV range between 300 and 400 nm was measured and was 39 watts/m².

The plants were treated in the two-leaf stage with an aqueous spray liquor comprising the suspension concentrates (SC) A17 to A33 or C1 from Examples 2-3 with an application rate of 10 g/ha of metaflumizone. Seven and ten days after treatment (DAT) the active compound activity which remained was determined with a bioassay on larvae of Spodoptera eridania (southern armyworm). For this, the larvae were brought into contact with the plants and the mortality was determined three days later (Table 3).

The experiment demonstrates that the insecticide is more stable to UV light in formulations comprising the UV absorbers tested than in formulations without UV absorber.

TABLE 3

| | |
|---|---|
| UV stabilizer | Mortality in [%] |

| SC | from Example | 7 DAT | 10 DAT |
|---|---|---|---|
| C1^{a)} | none | 55 | 22 |
| A18 | 1C | 100 | 96 |
| A19 | 1D | 74 | 70 |
| A32 | 1C | 100 | 84 |
| A33 | 1D | 100 | 41 |
| C4^{a)} | Uvinul ® P25 | 52 | 56 |

^{a)}Comparison experiment, not according to the invention

### Example 4

### Determination of the Surface Tension

The surface tension (ST) of the UV absorbers 1C and 1F was determined at 25° C. at concentrations of the UV absorber of from about 1 mg/l to 5,000 mg/l (Table 4). The ST of water to air is, for comparison, 72.4 mN/m, the ST of commercial surfactants is about 30-35 mN/m. The results show that the UV absorbers according to the invention can significantly lower the surface tension, i.e. that they are surface-active. They also show that they are able to reduce surface tension to a greater degree than commercially available UV absorbers. It was also shown that UV absorbers according to the invention are similar in surface activity to commercial surfactants.

TABLE 4

| UV absorber from | |
|---|---|
| Example | ST [mN/m] |
| Tinuvin ® 1130^{a)} | 40.3 |
| Uvinul ® P25^{a)} | 56.9 |
| 1C | 39.5 |
| 1F | 34.7 |

^{a)}not according to the invention

1-10. (canceled)

11. A composition comprising a pesticide and a UV absorber, wherein the UV absorber corresponds to the formula I 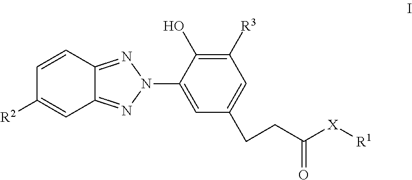
wherein X: NH or O;
R¹: —[C₂-C₄ alkoxy]ₙ—(C₁-C₁₈ alkyl) or —[CH₂CH₂NH]ₙ—H;
R²: H or Cl;
R₃: H or C₁-C₈ alkyl; and
n: 3 to 50.

12. The composition according to claim 11, wherein the pesticide is UV-sensitive.

13. The composition according to claim 11, wherein the weight ratio of the pesticide to UV absorber is from 30:1 to 1:3.

14. The composition according to claim 11, wherein n represents 3 to 15.

15. The composition according to claim 11, wherein the pesticide is a herbicide selected from the group consisting of napropamide, propanil, bentazone, paraquat dichloride, cycloxydim, sethoxydim, ethalfluralin, oryzalin, pendimethalin, trifluralin, acifluren, aclonifen, fomesafen, oxyfluoren, ioxynil, imazetapyr, imazaquin, chloridazon, norflurazon, thiazopyr, triclopyr, dithiopyr, diflufenican, picolinafen, amidosulfuron, molinate, vernolate, prometon, metribuzin, azafenidin, carfentrazone-ethyl, sulfentrazone, metoxuron, monolinuron, fluchloralin and flurenol; a fungicide selected from cyprodinil, fuberidazole, dimethomorph, procloraz, triflumizole, tridemorph, edifenfos, fenarimol, nuarimol, ethirimol, quinoxylen, dithianon, metominostrobin, trifloxystrobin, dichlofluamid, bromuconazole and myclobutanil; or an insecticide selected from acephate, azinphos-ethyl, azinphos-methyl, isofenphos, chlorpyriphos-methyl, dimethylvinphos, phorate, phoxim, prothiofos, cyhexatin, alanycarb, ethiofencarb, pirimicarb, thiodicarb, fipronil, bioallethrin, bioresmethin, deltamethrin, fenpropathin, flucythrinate, taufluvalinate, alpha-cypermethrin, metaflumizone, zeta-cypermethrin, resmethin, tefluthrin, lambda cyhalothrin and hydramethylnon.

16. The composition according to claim 11, wherein the pesticide is a pyrethroid.

17. The composition according to claim 11, wherein the pesticide is alpha-cypermethrin or metaflumizone.

18. The composition according to claim 11, wherein the composition comprises 0.5 to 30 wt % of UV absorber.

19. The composition according to claim 11, wherein the composition is a suspension.

20. The composition according to claim 11, wherein the UV absorber has a surface tension at the interface of water to air at 25° C. of at most 40 mN/m.

21. A UV absorber of the formula (III) 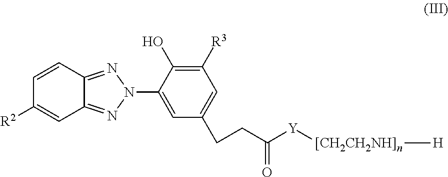
wherein Y: NH or O;
R²: H or Cl;
R₃: H or C₁-C₈ alkyl; and
n: 3 to 50.

22. The UV absorber according to claim 21, wherein n represents 3 to 15.

23. The UV absorber according to claim 21, wherein Y represents NH.

24. A method of controlling phytopathogenic fungi and/or undesirable plant growth and/or undesirable insect or mite infestation and/or of regulating plant growth, wherein the composition according to claim 11 is allowed to act on the particular pests, their habitat or the plants to be protected from the particular pest, the soil and/or on the undesirable plants and/or the crop plants and/or their habitat.

25. The method of claim 24, wherein the pesticide is UV-sensitive.

26. The method of claim 24, wherein the weight ratio of the pesticide to UV absorber is from 30:1 to 1:3.

27. The method of claim 24, wherein n represents 3 to 15.

28. The method of claim 24, wherein the pesticide is a herbicide selected from the group consisting of napropamide, propanil, bentazone, paraquat dichloride, cycloxydim, sethoxydim, ethalfluralin, oryzalin, pendimethalin, trifluralin, acifluren, aclonifen, fomesafen, oxyfluoren, ioxynil, imazetapyr, imazaquin, chloridazon, norflurazon, thiazopyr, triclopyr, dithiopyr, diflufenican, picolinafen, amidosulfuron, molinate, vernolate, prometon, metribuzin, azafenidin, carfentrazone-ethyl, sulfentrazone, metoxuron, monolinuron, fluchloralin and flurenol; a fungicide selected from cyprodinil, fuberidazole, dimethomorph, procloraz, triflumizole, tridemorph, edifenfos, fenarimol, nuarimol, ethirimol, quinoxylen, dithianon, metominostrobin, trifloxystrobin, dichlofluamid, bromuconazole and myclobutanil; or an insecticide selected from acephate, azinphos-ethyl, azinphos-methyl, isofenphos, chlorpyriphos-methyl, dimethylvinphos, phorate, phoxim, prothiofos, cyhexatin, alanycarb, ethiofencarb, pirimicarb, thiodicarb, fipronil, bioallethrin, bioresmethin, deltamethrin, fenpropathin, flucythrinate, taufluvalinate, alpha-cypermethrin, metaflumizone, zeta-cypermethrin, resmethin, tefluthrin, lambda cyhalothrin and hydramethylnon.

29. The method of claim 24, wherein the pesticide is a pyrethroid.

30. The method of claim 24, wherein the pesticide is alpha-cypermethrin or metaflumizone.
